(19) United States
(12) Patent Application Publication
(10) Pub. No.: US 2021/0369546 A1
(43) Pub. Date: Dec. 2, 2021

(54) Complementary Alternative Medicine Cold Compress Temple Massager

(71) Applicant: Joseph G. Meisch, Cazadero, CA (US)

(72) Inventor: Joseph G. Meisch, Cazadero, CA (US)

(21) Appl. No.: 15/929,981

(22) Filed: Jun. 1, 2020

(51) Int. Cl. A61H 7/00; A61F 7/02

(52) U.S. Cl. CPC A61H 7/007; A61F 7/02; A61H 2201/0153; A61H 2201/0107; A61F 2007/0279; A61H 2201/0214; A61F 2007/0203; A61H 2201/1695; A61F 2007/0269; A61H 2201/0192

(57) ABSTRACT

A temple massager including first and second flexible arcuate arms, each having a proximal portion connected to a handle and a distal portion, each of said distal portions having tip retention structure configured to capture and retain first and second pressure members; a selectively removable pressure member is disposed on each of the tip retention structures. The pressure members are fabricated from cold compress material and may include a compartment for containing a fluid or gel cold compress composition. A tension adjustment mechanism is disposed between the arcuate arms proximate the handle to adjust the spacing between the pressure members and thereby adjust pressure applied to a user's temples.

### CROSS REFERENCES TO RELATED APPLICATIONS

The present application claims the benefit of the filing date of U.S. Provisional Patent Application Ser. No. 62/854,906, filed May 30, 2019 (May 30, 2019).

### STATEMENT REGARDING FEDERALLY SPONSORED RESEARCH OR DEVELOPMENT

Not applicable.

### THE NAMES OR PARTIES TO A JOINT RESEARCH AGREEMENT

Not applicable.

### INCORPORATION BY REFERENCE OF MATERIAL SUBMITTED ON A COMPACT DISC

Not applicable.

### SEQUENCE LISTING

Not applicable.

### BACKGROUND OF THE INVENTION

Field of the Invention: The present invention relates most generally to massage apparatus, and more particularly to hand held self-massage apparatus, and more particularly still to a complementary alternative medicine hand held temple massager with selectively attachable/detachable cold elements fabricated from or containing a refrigerant that absorbs heat from a user's temples during use.

### BACKGROUND DISCUSSION

It is well known that tension headaches can be alleviated, at least in part, by massaging the scalp and/or the temples. Unfortunately, self-massage is virtually useless, as it introduces nearly as much tension as it relieves; indeed, the muscular effort required to elevate the arms and exert pressures on the temples can actually increase tension in the neck and shoulders, which in some instances is the cause of a tension headache. Finding a ready, willing, and able masseur/masseuse to take on the task when needed is expensive or too time-consuming to be of any use.

For these reasons, self-administered scalp and temple massagers have been devised to provide a headache sufferer the means to apply headache-reducing pressure to his or her own temples. Further, it well known to use cold and/or hot compresses to treat headaches, both tension headaches and migraine headaches. Presently, even the National Headache Foundation and the Mayo Clinic recommend the use of cold compresses and ice packs to treat migraine headaches. How cold and heat relieve headache pain is poorly understood, though anecdotal evidence of efficacy is overwhelming. Clinicians continue to look at vascular, neurological, and endocrine system contributions. Despite the lack of definitive studies showing efficacy, early studies show considerable promise.

Accordingly, it would be useful to provide a combination massage device and cold or hot compress for self-treatment of headache pain. To that end, the present invention is an improvement on the temple massager earlier invented by the present inventor and described in U.S. Pat. No. 7,399,287 (“Meisch '287”), which is incorporated in its entirety by reference herein, the improvement comprising selectively replaceable cold compress tips fabricated from and/or containing a refrigerant substance that may be refrigerated until sufficiently cold to provide sought-after headache relief.

The temple massager of Meisch '287 stimulates facial muscles nearly instantaneously releasing tension. The massager enables a user to engage and stimulate the trigeminal nerve, and thereby to self-trigger the “relaxation response.” Its efficacy has been documented anecdotally in Afghanistan by combat service members suffering either or both periodic tension headaches from PTSD and chronic headaches from traumatic brain injury (TBI).

### SUMMARY OF THE INVENTION

The present invention represents and advancement and an improvement on Meisch '287, at least for some applications. The temple massager of the present invention is configured for selective installation and removal of replaceable cold compress tips. The tips include a refrigerant, such as a refrigerant gel, and may be refrigerated by placement in a freezer. Sized for heat transfer over a sufficiently long period of time, the replaceable tips have sufficient mass and a suitable size and shape to function as effective massaging tips and cold compresses, providing a calming effect and preventing and reducing headache pain, especially pain from temporomandibular joint disorders (TMD, TMJ).

### BRIEF DESCRIPTION OF SEVERAL VIEWS OF THE DRAWINGS

FIG. 1A is an upper perspective view of the temple massager of the present invention, showing the removable cold compress tips aligned for insertion on the distal ends of the respective flexible arcuate arms of the massager;

FIG. 1B is the same view showing the tips installed on the ends of the arms;

FIG. 2A is a detailed top view of the distal end of the first arm with the replaceable cold compress tip poised for installation;

FIG. 2B is the same view with the replaceable tip installed on the arm;

FIG. 3 is a cross-sectional side view in elevation as taken along section line 3-3 of FIG. 2B; and

FIG. 4 is a side view in elevation as seen from view line 4-4 of FIG. 2B.

### DETAILED DESCRIPTION OF THE INVENTION

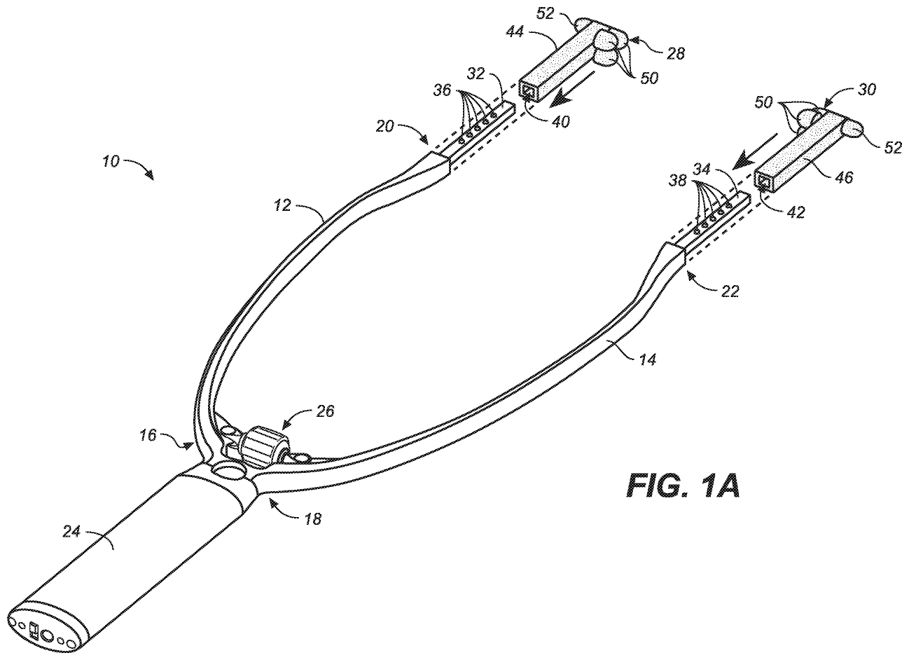

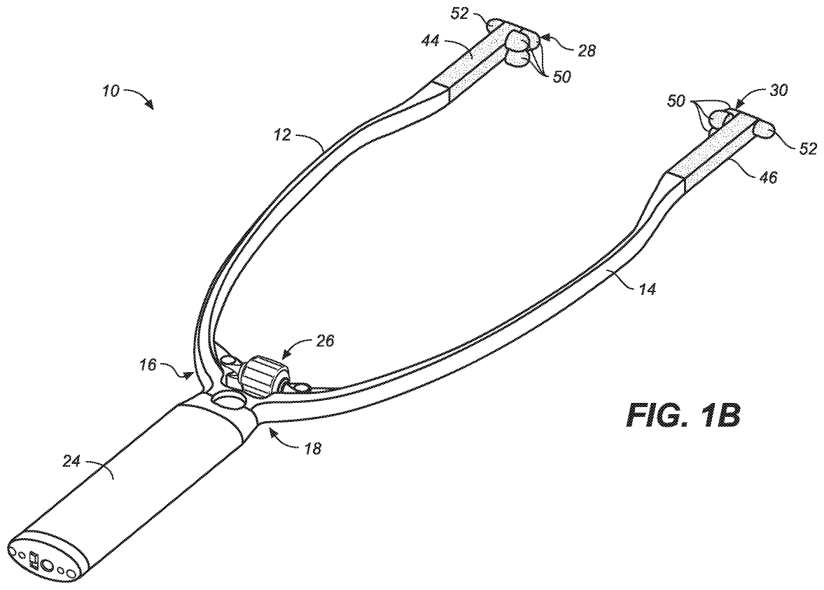

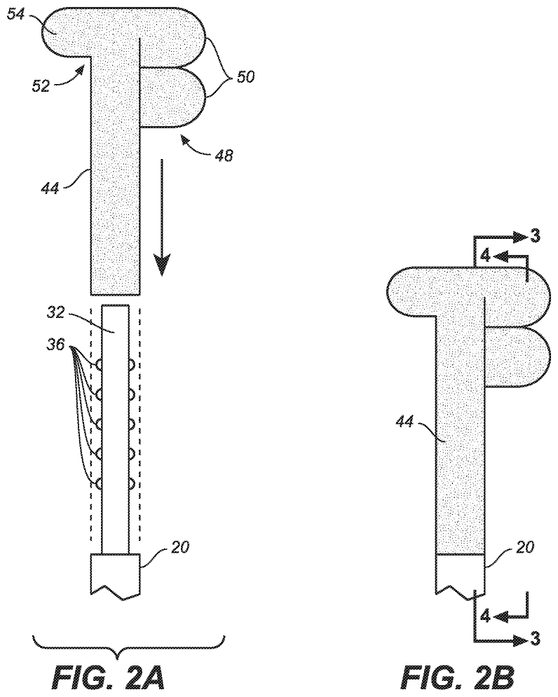

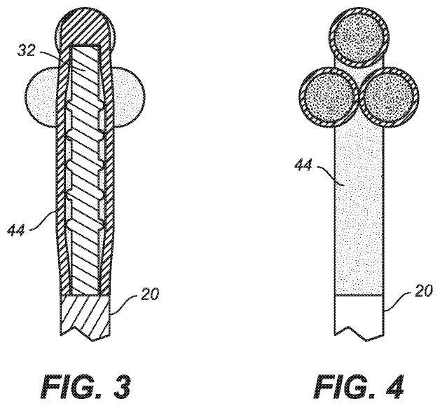

Referring to FIGS. 1A through 4, there is illustrated therein a new and improved complementary alternative medicine temple massager with selectively removable cold compress tips, the invention generally denominated 10 herein.

In an embodiment, the massager comprises first and second flexible arcuate arms 12, 14, respectively, each having a proximal portion 16, 18, and a distal portion 20, 22. The proximal portions terminate in a connection to a handle 24, which is preferably integral with the proximal ends, such that the arms lie in substantially the same plane. A pressure or tension adjustment mechanism—for instance, a turnbuckle 26—is disposed between the first and second arcuate arms proximate the handle in the proximal portion of the arcuate arms. This tensioning mechanism enables users to adjust the spacing between the distal portions of the arcuate arms so that pressure members 28, 30 disposed on the distal portion of the flexible arms engage the user's head with a desired pressure.

Each of the first and second arcuate arms includes structure for capturing and retaining first and second pressure members, 28, 30, respectively, which are employed to apply pressure to the temple areas of a user during use. In an embodiment, this may comprise a male element 32, 34 integrally formed in the distal portions of the flexible arcuate arms and having surface features (e.g., nubs) 36, 38, to provide a slidable friction fit with female sockets 40, 42 in respective removable sleeves 44, 46. The sleeves, in turn, may be integrally formed with the pressure members 28, 30. The sleeves are preferably slightly resilient so as to facilitate both slidable insertion and releasable capture of the male elements. In this manner, the pressure members are essentially snapped onto each arm and can be retained until removal is desired.

In another embodiment, the male/female configurations for the pressure fit connection of the pressure elements to the massager arms can be reversed. Nubs on the male element—whether on the distal portions of the flexible arcuate arms or on the removable pressure elements—preferably project out about 1/16th of an inch. Sizing can be tailored to the clearances between the female sleeve and the male portion, as well as to the resilience of the coupling materials.

In embodiments, alternative coupling structures can be employed to effect cantilevered snap fit connections, nipple-and-hole connections, tab-and-slot connections, simple friction fit connections, pin-and-socket connections, and the like. As will be readily appreciated, the objective is to provide structure for an easy and quick coupling between the massager arms and the pressure elements.

In embodiments, the inboard portion 48 of the pressure members are configured with surface features, such as the three hemispherical surfaces 50 shown on pressure elements 28 and 30. Countless alternative shapes can be conceived and implemented that are conducive to applying effectively focused pressure. The outboard portion 52 of the pressure elements may comprise a hollow compartment for containing a fluid or gel cold compress composition.

Cold compress materials for at least the tip portion of the removable pressure elements may include any of a number of solid thermoplastic materials or materials encasing a cold retaining composition suitable for extended transfer of heat from a user's head to the pressure elements when refrigerated. Such compositions may include water with added hydroxyethyl cellulose, silica gel, ammonium nitrate, urea, sodium polyacrylate, ammonium chloride, or diethylene glycol, all of which may be coated with vinyl or another chemically inert material barrier. In embodiments in which an interior volume is required for holding solid or semi-solid gel-like composition, testing shows that an approximately 2 inch by 1¼ body is sufficient for effective thermal transfer to reduce headache. Sizes may range, however, depending on the materials for the cold composition and the desired time of use.

In another embodiment, the entire tip is a freezable tip fabricated from a combination of rubber and plastic, such as SANTOPRENE®. [SANTOPRENE is a registered trademark of Exxon Mobile Corporation of Irving, Tex.]

As can be seen from the foregoing, in its most essential aspect, the temple master of the present invention includes first and second flexible arcuate arms, each having a proximal portion connected to a handle and a distal portion, each distal portion having tip retention structure configured to capture and retain the first and second pressure members. Selectively removable and replaceable pressure members fabricated from cold compress material are disposed on each tip retention structure. A tension adjustment mechanism is disposed between the arcuate arms proximate the handle to adjust the spacing between the pressure members and thereby adjust pressure applied to a user's temples.

What is claimed as invention is:

1. A temple massager, comprising:
first and second flexible arcuate arms, each having a proximal portion connected to a handle and a distal portion, each distal portion having tip retention structure configured to capture and retain first and second selectively removable pressure members;
first and second selectively removable pressure members, one each disposed on said tip retention structure, said pressure members fabricated from cold compress material; and
a tension adjustment mechanism disposed between said arcuate arms proximate said handle to adjust the spacing between said tips and thereby adjust pressure applied to a user's temples.

2. The temple massager of claim 1, wherein said pressure members each comprise removable sleeves with surface features and a female socket, and wherein said tip retention structure includes a male element integrally formed in said distal portions of each of said flexible arcuate arms and has surface features to provide a slidable friction fit with said female sockets in said respective removable sleeves.

3. The temple massager of claim 2, wherein said removable sleeves are integrally formed with said pressure members.

4. The temple massager of claim 3, wherein said removable sleeves are resilient so as to facilitate both slidable insertion and releasable capture of the male elements.

5. The temple massager of claim 2, wherein said removable sleeves are resilient so as to facilitate both slidable insertion and releasable capture of the male elements.

6. The temple massager of claim 1, wherein said selectively removable pressure members each include a male element with surface features, and said distal portions of said arcuate arms include a female socket for slidable insertion of said male element to provide a slidable friction fit of said male element and said female sockets.

7. The temple massager of claim 6, wherein said removable sleeves are integrally formed with said pressure members.

8. The temple massager of claim 7, wherein said removable sleeves are resilient.

9. The temple massager of claim 1, wherein said tip retention structure and said pressure members are configured with complementary coupling structure selected from the group consisting of nipple-and-hole connectors, tab-and-slot connectors, friction fit connectors, and pin-and-socket connectors.

10. The temple massager of claim 1, wherein said pressure members include an inboard portion having surface features to focus pressure applied by said massager.

11. The temple massager of claim 10, wherein said surface features include a plurality of hemispherical surfaces.

12. The temple massager of claim 11, wherein said pressure members include an outboard portion having a hollow compartment for containing a fluid or gel cold compress composition.

13. The temple massager of claim 1, wherein said pressure members include an outboard portion having a hollow compartment for containing a fluid or gel cold compress composition.

14. The temple massager of claim 1, wherein said pressure members are fabricated from a solid thermoplastic material or combination of materials encasing a cold-retaining composition.

15. The temple massager of claim 14, wherein said cold-retaining composition is selected from the group consisting of water with added hydroxyethyl cellulose, silica gel, ammonium nitrate, urea, sodium polyacrylate, ammonium chloride, and diethylene glycol.
